(19) United States
(12) Patent Application Publication
(10) Pub. No.: US 2012/0165607 A1
(43) Pub. Date: Jun. 28, 2012

(54) GUIDE ASSEMBLY FOR ENDOSCOPE

(76) Inventors: Tsuyoshi Ashida, Kanagawa (JP); Takayuki Nakamura, Kanagawa (JP); Shinichi Yamakawa, Kanagawa (JP); Yasunori Ohta, Kanagawa (JP); Masayuki Iwasaka, Kanagawa (JP)

(21) Appl. No.: 13/336,715

(22) Filed: Dec. 23, 2011

(30) Foreign Application Priority Data
Dec. 24, 2010 (JP) 2010-287357

(51) Int. Cl. A61B 1/00

(52) U.S. Cl. 600/114

(57) ABSTRACT

A guide assembly for an endoscope having an elongated tube includes a first inner sleeve, a first outer sleeve, an endless track device, engagement rollers, a second inner sleeve and idler rollers. The endless track device has an annular surface, is supported around the first outer sleeve, for endlessly moving in an axial direction of the elongated tube in contact with a wall of the body cavity, for propulsion of the elongated tube. A setting device as second outer sleeve is shiftable between a set position and a release position, for pressing the idler roller on the engagement roller when in the set position, to nip the endless track device therewith, and for retracting the idler roller from the engagement roller in an outward direction when in the release position, to facilitate removal of the endless track device from the first outer sleeve.

### BACKGROUND OF THE INVENTION

1. Field of the Invention

The present invention relates to a guide assembly for an endoscope. More particularly, the present invention relates to a guide assembly in which a propulsion unit with an endless track device can be easily removed from a transmission device for driving the propulsion unit by attachment to the endoscope.

2. Description Related to the Prior Art

An endoscope has an elongated tube or guide tube for entry in a body cavity of a patient. The elongated tube includes ahead assembly, a steering device and a flexible tube. The head assembly is positioned at its tip. The steering device steers the head assembly in a direction as desired. The flexible tube extends from the steering device. An imaging window and the like are formed in a distal end of the head assembly for imaging of an object.

Manipulation of the endoscope is a difficult process, because a large intestine in a gastrointestinal tract is a tortuous organ in a human body, and some body parts are very changeable in the position in the body. Learning the manipulation of the endoscope of the large intestine requires much experience and time. If a doctor is insufficiently skilled in the manipulation, physical load to the body of a patient will be very large.

Portions of the large intestine where entry of an endoscope is locally very difficult include a sigmoid colon and transverse colon. Specifically, the sigmoid colon and transverse colon is present in the body at changeable locations unlike other body parts. Their shapes are changeable within ranges of their sizes. Also, the sigmoid colon and transverse colon may be deformed in the body by contact pressure of the endoscope upon its entry. Various ideas of manipulation have been suggested for enabling straight forms of the sigmoid colon or the transverse colon so as to reduce contact a wall of the large intestine upon entry of the endoscope. In view of this, there is a suggestion of a guide assembly or self-propelled apparatus as a structure featured today, which propels the endoscope in a distal direction in an intestine or colon in order to facilitate the entry even with manipulation of an unskilled operator.

The guide assembly disclosed in U.S. Pat. Nos. 6,971,990 and 7,736,300 (corresponding to JP-A 2009-513250) includes a housing, a movable endless track device or crawler device or toroidal device, and a transmission device or driving device. The housing is a sleeve for receiving insertion of an elongated tube of the endoscope. The endless track device is disposed to extend around the housing. The transmission device endlessly turns around the endless track device to move the endoscope in the distal or proximal direction as axial direction. The endless track device is formed from resilient material, and is filled with fluid (liquid or gas) for inflation in a manner of a balloon of rubber, for contact with a wall of the intestine at a conditioned pressure. The endoscope can move smoothly through the intestine in contact with the wall.

The endoscope after the use at one time is cleaned up by a washer constructed specially. The guide assembly must be removed from the endoscope and washed. However, the guide assembly according to U.S. Pat. Nos. 6,971,990 and 7,736,300 must be disassembled for washing, because an outer unit including the endless track device to move in contact with the wall is pressed on an inner unit for attachment to the elongated tube by a pair of rollers which squeeze the endless track device in inward and outward directions. Although the endless track device should be exchanged at each time of the use after direct contact with the wall, it is important to reuse the inner unit by washing because of incorporation of the transmission device.

### SUMMARY OF THE INVENTION

In view of the foregoing problems, an object of the present invention is to provide a guide assembly in which a propulsion unit with an endless track device can be easily removed from a transmission device for driving the propulsion unit by attachment to the endoscope.

In order to achieve the above and other objects and advantages of this invention, a guide assembly for an endoscope having a section of an elongated tube for entry in a body cavity is provided. A first inner sleeve has a lumen, for mounting on the elongated tube by receiving entry in the lumen. A first outer sleeve is secured around the first inner sleeve. An endless track device has an annular surface, is supported around the first outer sleeve, for endlessly moving in an axial direction of the elongated tube in contact with a wall of the body cavity, for propulsion of the elongated tube. An engagement roller is secured to the first outer sleeve in a rotatable manner, for driving the endless track device endlessly to move. A second inner sleeve is disposed in an inner space within the endless track device. An idler roller is secured to the second inner sleeve, and caused to rotate by endless movement of the endless track device. A setting device is shiftable between a set position and a release position, for pressing the idler roller on the engagement roller when in the set position, to nip the endless track device therewith, and for retracting the idler roller from the engagement roller in an outward direction when in the release position, to enable removal of the endless track device from the first outer sleeve.

The setting device includes a second outer sleeve disposed in the inner space within the endless track device and around the second inner sleeve, for rotating relative to the second inner sleeve between the set position and the release position.

The first inner sleeve and the first outer sleeve constitute a unit for transmission, and the second inner sleeve, the endless track device and the setting device constitute a toroidal propulsion unit.

The second outer sleeve includes a pressure portion for pressing the idler roller on the engagement roller when in the set position. A release portion enables the idler roller to move away from the engagement roller when in the release position.

The pressure portion is an internal pressure surface of the second outer sleeve, and the release portion is a release opening or recess for receiving the idler roller.

The setting device further includes a biasing mechanism for biasing the idler roller away from the engagement roller.

A set of the engagement roller and the idler roller is constituted by plural sets arranged equidistantly in a circumferential direction of the first outer sleeve and the second inner sleeve and by plural sets arranged in the axial direction.

The idler roller is constituted by two idler rollers disposed on distal and proximal sides relative to the engagement roller in the axial direction.

The biasing mechanism is a spring plate retained on the second inner sleeve.

Furthermore, a front end ring is secured to distal ends of the first inner sleeve and the first outer sleeve. A rear end ring is secured to proximal ends of the first inner sleeve and the first outer sleeve, for retaining the first inner sleeve and the first outer sleeve together with the front end ring.

The front and rear end rings are formed together with the first outer sleeve.

Furthermore, an input gear is contained in the first outer sleeve, and caused to rotate by an external drive source. A gear mechanism is disposed between the first inner sleeve and the first outer sleeve, for rotating the engagement roller upon rotation of the input gear.

The gear mechanism includes a spur gear of a sleeve shape, supported around the first inner sleeve in a rotatable manner, and meshed with the input gear. Worm gear teeth are formed with a distal end of the spur gear, and helically engaged with the engagement roller.

Furthermore, a rear end ring is secured to proximal ends of the first inner sleeve and the first outer sleeve, for retaining the first inner sleeve and the first outer sleeve, and for supporting the input gear in a rotatable manner.

Furthermore, a torque coil structure is connected with the drive source, for applying rotational force to the input gear.

The idler roller is constituted by first and second idler rollers having rotational axes substantially parallel with one another. Furthermore, a roller support device supports the first and second idler rollers in a rotatable manner.

The biasing mechanism extends from the roller support device in the axial direction and crosswise to the first idler roller. The first idler roller includes first and second rolls, positioned at respectively roller ends, and supported by the roller support device. A center portion is disposed to extend between the first and second rolls, has a smaller diameter than the first and second rolls, for allowing the biasing mechanism to shift.

The roller support device includes first and second side walls, opposed to one another, for supporting the first and second idler rollers in a rotatable manner. An arm is disposed between the first and second idler rollers, for connecting the first side wall with the second side wall and supporting the first and second side walls, the biasing mechanism being retained on the arm.

Accordingly, a propulsion unit with an endless track device can be easily removed from a transmission device for driving the propulsion unit by attachment to the endoscope, because the setting device is movable to the release position for facilitating the removal of the endless track device.

### BRIEF DESCRIPTION OF THE DRAWINGS

The above objects and advantages of the present invention will become more apparent from the following detailed description when read in connection with the accompanying drawings, in which:

FIG. 1 is a plan illustrating an endoscope;

FIG. 2 is a vertical section illustrating a guide assembly for the endoscope;

FIG. 3 is an exploded perspective view illustrating the guide assembly;

FIG. 4 is an exploded perspective view illustrating a drive unit;

FIG. 5 is a front elevation illustrating a roller support device;

FIG. 6 is a vertical section illustrating the roller support device;

FIG. 7 is a vertical section, partially broken, illustrating the guide assembly in enlargement;

FIG. 8 is a vertical section, partially broken, illustrating a combination of a second inner sleeve and a second outer sleeve in enlargement;

FIG. 9 is a cross section, partially broken, illustrating engagement between an engagement roller and idler roller;

FIG. 10 is a cross section, partially broken, illustrating the same as FIG. 9 but in a release position with a phase difference of 40 degrees;

FIG. 11 is a vertical section illustrating the guide assembly in the release position.

### DETAILED DESCRIPTION OF THE PREFERRED EMBODIMENT(S) OF THE PRESENT INVENTION

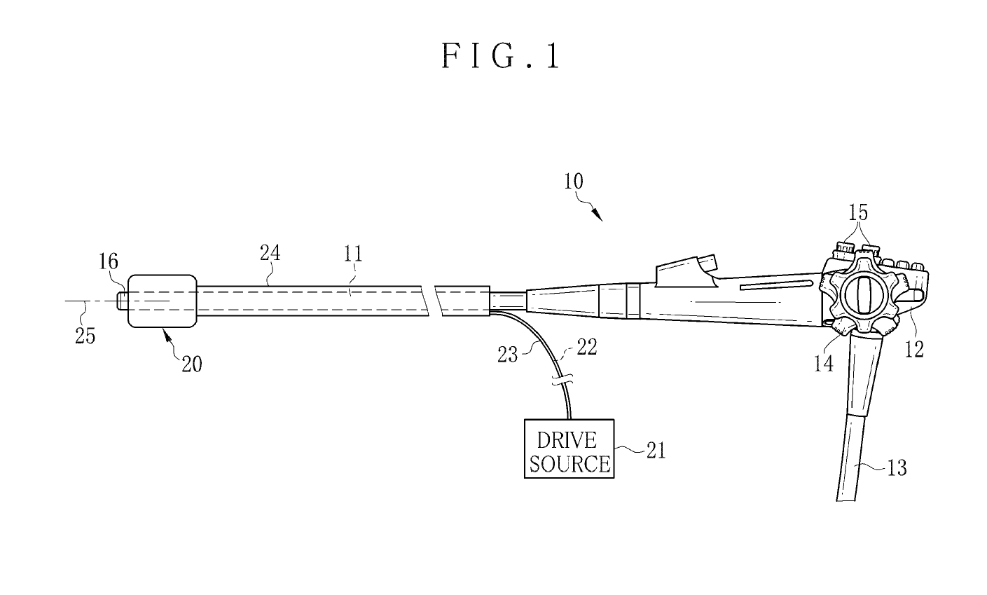

In FIG. 1, an endoscope 10 is an electronic instrument, of which a head assembly includes a small type of CCD sensor or CMOS sensor as image pickup device or image sensor. The endoscope 10 includes a section of an elongated tube 11 or guide tube, a handle device 12, and a universal cable 13. The elongated tube 11 is provided with the image pickup device, and is entered in a body cavity, for example, a large intestine in a gastrointestinal tract. The handle device 12 is manipulated by a doctor or operator to hold the endoscope 10 and control the elongated tube 11. The universal cable 13 connects the endoscope 10 to a processing apparatus and a light source apparatus (not shown). The universal cable 13 is connected to the handle device 12, and includes a fluid supply channel, a signal line for an image signal, and a light guide device as optics. Also, the handle device 12 includes a set of steering wheels 14 and an air/water button 15.

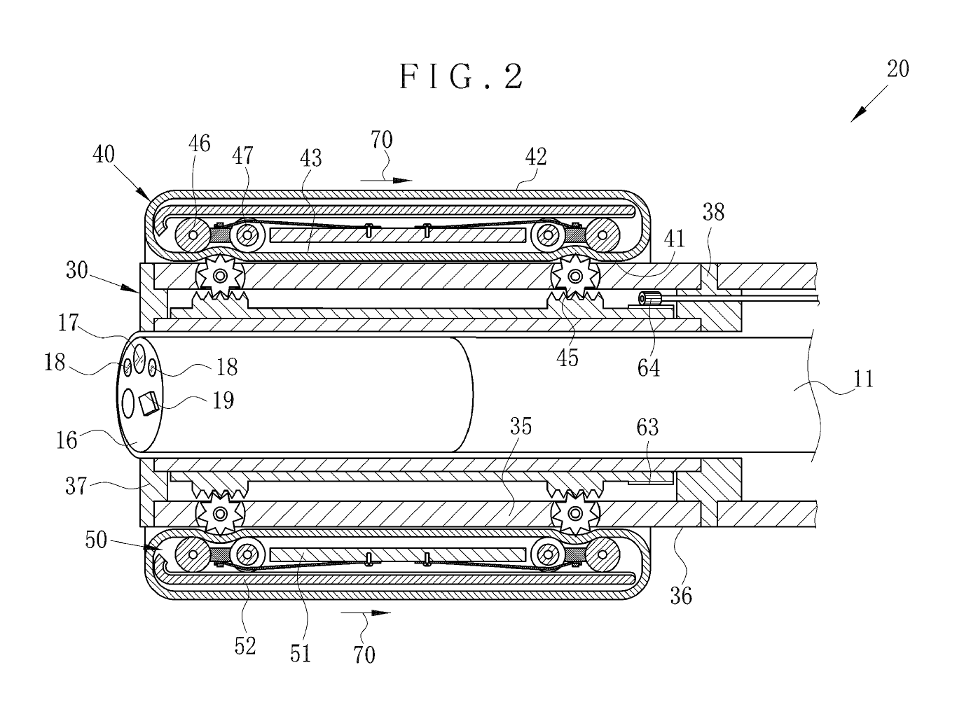

The steering wheels 14 are rotated for adjusting the elongated tube 11 for its steering direction and amount of steering. The air/water button 15 is depressible for supply of air and water or suction. The elongated tube 11 is a flexible tube, and has a head assembly 16. Various openings are formed in a distal end of the head assembly 16, including an imaging window 17, a lighting window 18 and an air/water nozzle 19. See FIG. 2. A guide assembly 20 or self-propelled assembly is mounted on the head assembly 16 of the elongated tube 11. The guide assembly 20 operates for moving the elongated tube 11 in a distal or proximal direction in the gastrointestinal tract.

A drive source 21 or motor drives the guide assembly 20. A torque coil structure 22 of a multi-component type is connected with the drive source 21, and transmits torque for moving the guide assembly 20. A protection sheath 23 fully covers the torque coil structure 22 for protection. The torque coil structure 22 is caused to rotate in the protection sheath 23 by the drive source 21. A control unit (not shown) controls the drive source 21. A button panel is connected with the control unit. The button panel includes an input button for inputting instruction signals for advance, return and stop of the guide assembly 20. A changing button in the button panel is operable for changing a moving speed of the guide assembly 20.

An overtube 24 is disposed to receive insertion of the elongated tube 11. The protection sheath 23 is positioned to extend between the overtube 24 and the elongated tube 11. The overtube 24 has a structure of bellows, and is compressible and expandable in an axial direction 25 of the elongated tube 11.

In FIG. 2, the guide assembly 20 includes a movable endless track device 40 or crawler device or toroidal device, and a transmission device 30 or sleeve device. The endless track device 40 is formed from a material of a sheet or film, has flexibility and softness, and is shaped in a toroidal form. The transmission device 30 receives entry of the head assembly 16 of the elongated tube 11 of the endoscope 10, is secured to the head assembly 16, and is entered in a lumen opening of the endless track device 40. The endless track device 40 has a lumen wall 41 of the lumen opening in contact with the transmission device 30, and is pushed and moved in a distal direction which is forward in the axial direction 25 of the endoscope 10. Thus, an outer wall 42 of the endless track device 40 in contact with a wall of the body cavity is moved in a proximal direction 70 which is opposite to the distal direction. The endless track device 40 is turned around continuously.

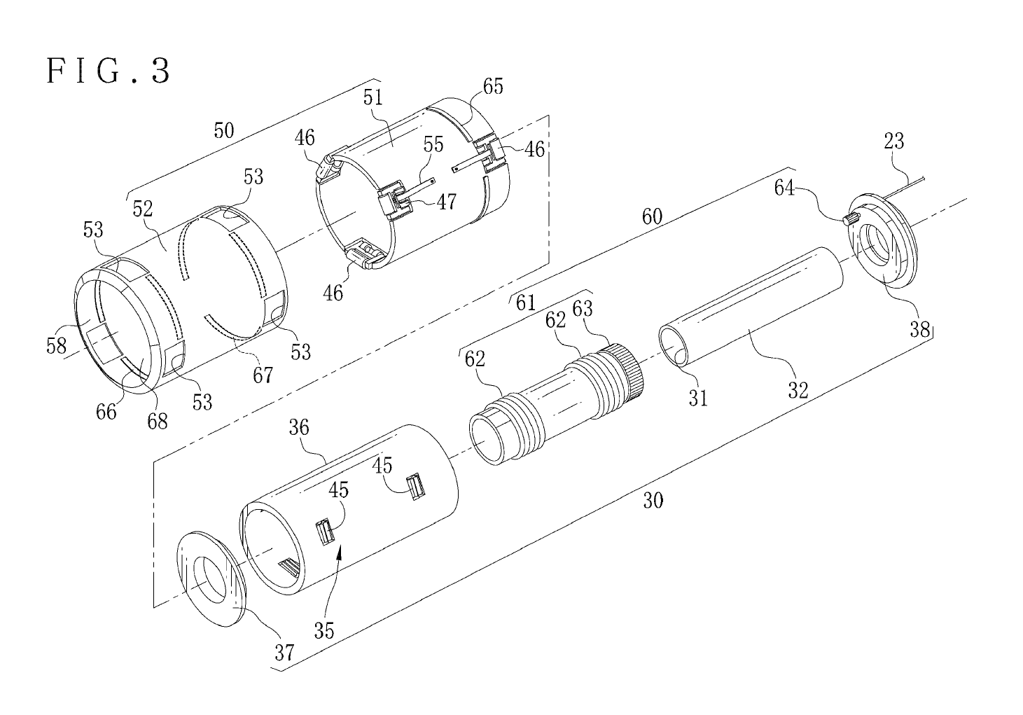

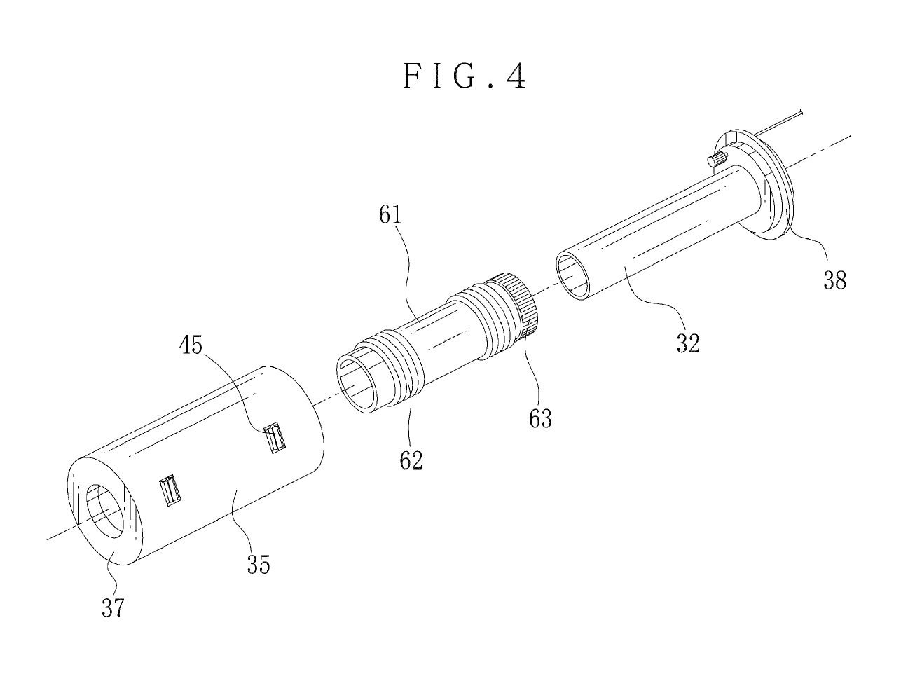

In FIGS. 2 and 3, the transmission device 30 includes a first inner sleeve 32 (shaft sleeve), a first outer sleeve 35 (support sleeve), a front end ring 37 and a rear end ring 38. A lumen 31 or through passage is formed through the first inner sleeve 32, and receives entry of the head assembly 16 of the elongated tube 11 of the endoscope 10, so that the first inner sleeve 32 is secured to the head assembly 16. The first outer sleeve 35 is disposed around and supported by the first inner sleeve 32. The front and rear end rings 37 and 38 are fitted on both of the first inner sleeve 32 and the first outer sleeve 35, and support those coaxially with an inner space of an annular shape. Note that it is possible as illustrated in FIG. 4 to form the front end ring 37 together with the first outer sleeve 35 and form the rear end ring 38 together with the first inner sleeve 32.

The first outer sleeve 35 has an outer surface 36. Plural engagement rollers 45 or drive rollers or toothed rollers (worm wheels) are disposed on the outer surface 36. The endless track device 40 has the lumen wall 41. A second inner sleeve 51 or housing sleeve is a component of a propulsion unit 50 or endless track unit. Idler rollers 46 and 47 or driven rollers are supported by the second inner sleeve 51. Each of the engagement rollers 45 nips the endless track device 40 in cooperation with the idler rollers 46 and 47. The lumen wall 41 of the endless track device 40 is moved in the distal direction (axial direction 25), to turn around the endless track device 40. The number of the engagement rollers 45 is six, including two arranged on the proximal and distal side of the first outer sleeve 35, and three arranged circumferentially on the first outer sleeve 35. Spur gear teeth are formed with the engagement roller 45. A worm gear 62 (worm) or worm gear teeth or worm thread is meshed with the spur gear teeth. To this end, the spur gear teeth are directed to the inside of the first outer sleeve 35.

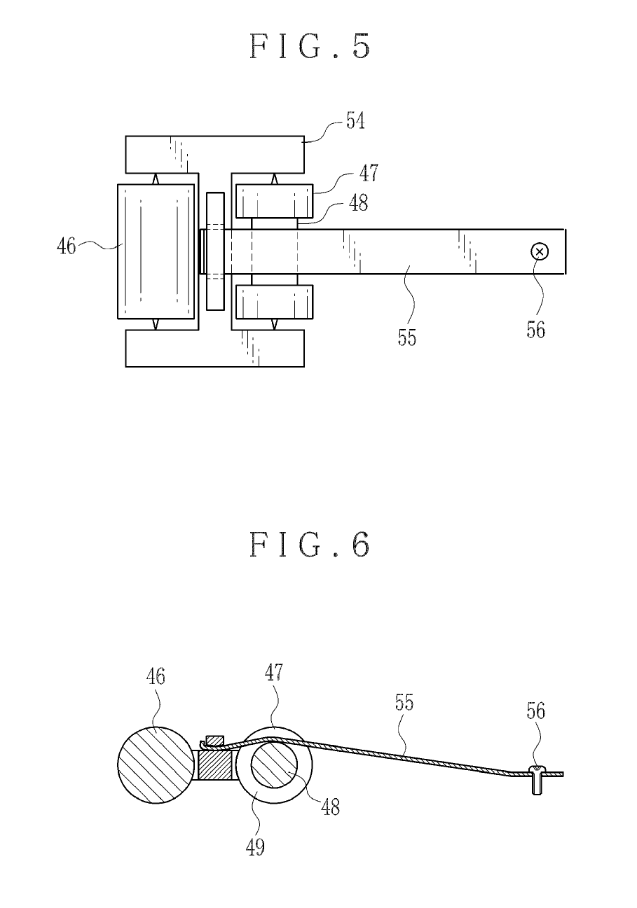

The propulsion unit 50 includes a second outer sleeve 52 or release sleeve as setting device, and the second inner sleeve 51. The second inner sleeve 51 is disposed in the inner space of the endless track device 40. Idler rollers 46 and 47 or driven rollers are supported on the second inner sleeve 51 in a rotatable manner. An inner surface 43 or channel surface of the endless track device 40 contacts the idler rollers 46 and 47, which squeeze the endless track device 40 in cooperation with the engagement roller 45. The second outer sleeve 52 is disposed around and supported by the second inner sleeve 51 in a rotatable manner. In FIGS. 5 and 6, a roller support device 54 has two side walls and an arm, and axially supports the idler rollers 46 and 47 in parallel with one another. A release spring 55 as a biasing mechanism in the setting device has one end with which the arm of the roller support device 54 is engaged between the idler rollers 46 and 47. A center portion 48 of the idler roller 47 has a smaller diameter, so that a space 49 for loosening or play is defined for shift of the release spring 55.

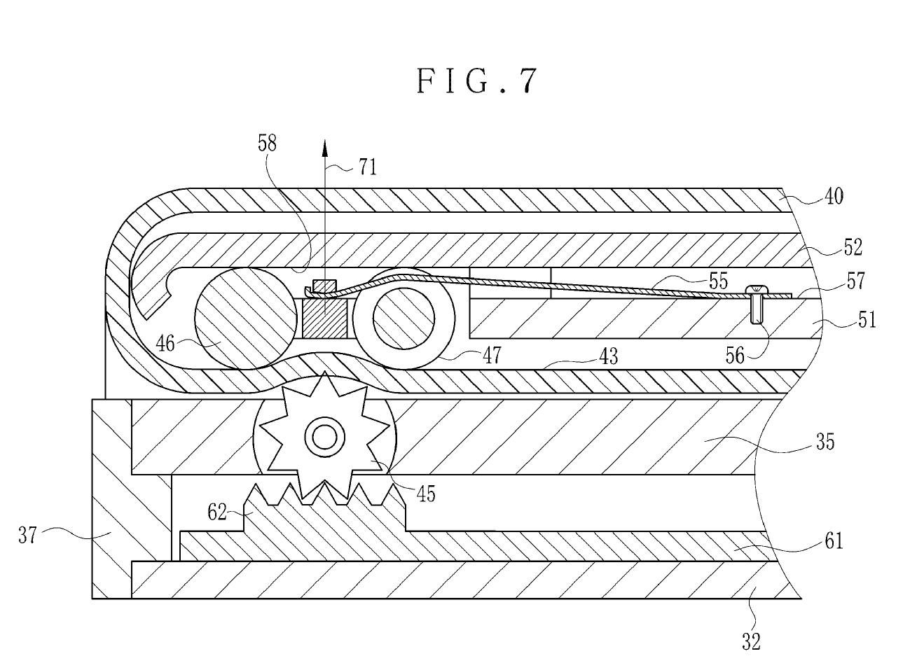

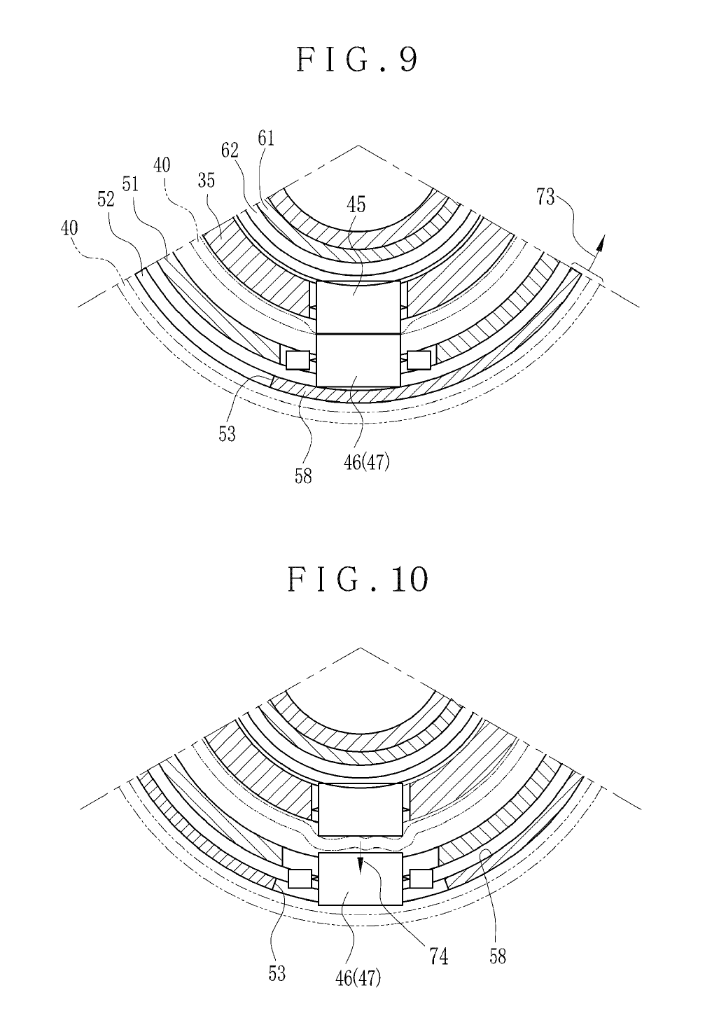

In FIGS. 3 and 7, there is an outer surface 57 of the second inner sleeve 51. A screw 56 is used to retain the idler rollers 46 and 47 to the outer surface 57 together with the release spring 55. The idler rollers 46 and 47 are supported in a rotatable manner in the axial direction of moving the endless track device 40, and also are biased by the release spring 55 in an outward direction 71, namely away from the engagement roller 45. The idler rollers 46 and 47 are arranged in the axial direction. When the second outer sleeve 52 is in a set position of FIG. 9, a pressure surface 58 inside the second outer sleeve 52 presses the idler rollers 46 and 47, which nip the endless track device 40 in directions with an inclination together with the engagement roller 45.

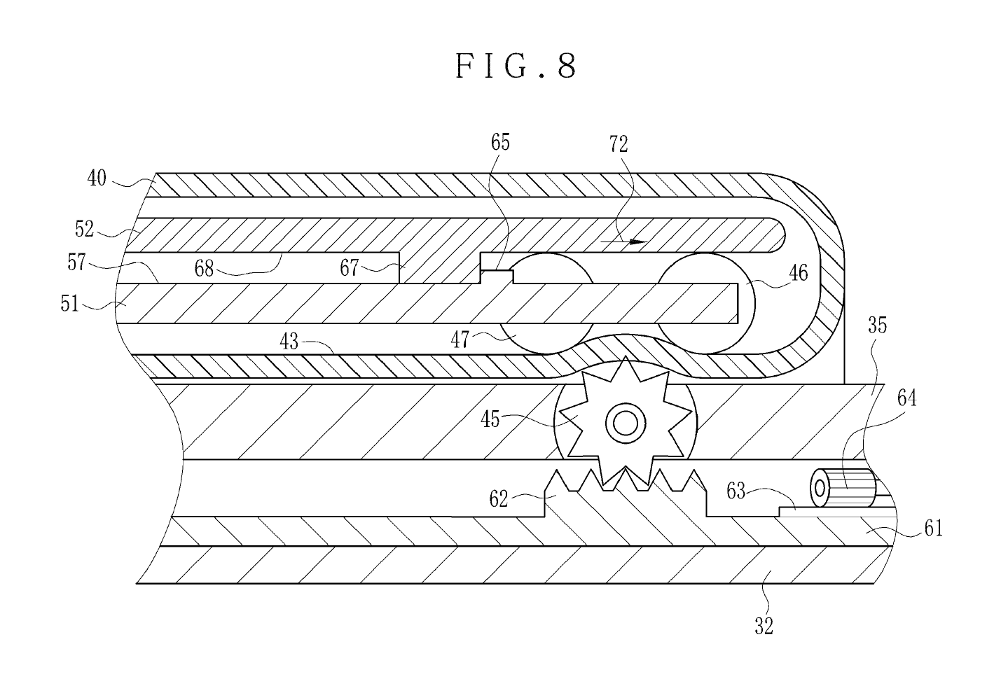

In FIGS. 3 and 8, the second outer sleeve 52 is combined with the second inner sleeve 51 by movement in a proximal direction. The second outer sleeve 52 has an inner surface 68. Engagement ridges 66 and 67 are formed on the inner surface 68, and include three arranged equidistantly on the circumference. The engagement ridges 66 are disposed near to a distal end of the inner surface 68. The engagement ridges 67 are disposed near to a proximal end of the inner surface 68. A stopper 65 is disposed at a proximal end of the outer surface 57 of the second inner sleeve 51, and becomes engaged with the engagement ridges 67 upon assembling the second outer sleeve 52 on the second inner sleeve 51. So the second outer sleeve 52 can be positioned exactly on the second inner sleeve 51. An arrow 72 indicates a direction (the proximal direction 70) in which the second inner sleeve 51 receives force of friction when the idler rollers 46 and 47 rotate. The second outer sleeve 52 does not move reverse to the outward direction 71.

In FIG. 3, release openings 53 as setting device are formed in the second outer sleeve 52. Each of the release openings 53 is formed between two portions of the pressure surface 58. When the second outer sleeve 52 is in the release position of FIG. 10, the idler rollers 46 and 47 enter the release opening 53 to retract those from the engagement roller 45. Pairs of the idler rollers 46 and 47 are arranged at three points arranged equidistantly on the circumference, and disposed at the proximal and distal end portions of the second inner sleeve 51. Note that a release recess can be formed instead of the release opening 53.

A gear mechanism 60 for driving is incorporated in the transmission device 30, and transmits rotation of the engagement roller 45. The gear mechanism 60 includes a threaded drive sleeve 61 or worm drive, a spur gear 63, and an input gear 64 or pinion. The drive sleeve 61 is supported axially by the first inner sleeve 32 in a rotatable manner. The input gear 64 is positioned on the rear end ring 38 in a rotatable manner. Two tooth sets of teeth of the worm gear 62 are disposed on the drive sleeve 61. The spur gear 63 or driven gear of a sleeve shape is disposed at a proximal end of the drive sleeve 61. The input gear 64 is disposed between the first inner sleeve 32 and the first outer sleeve 35, and meshed with the spur gear 63. The torque coil structure 22 is inserted through a hole in the rear end ring 38, is connected to the input gear 64, and transmits rotation to the worm gear 62.

Accordingly, a proximal portion of the endless track device 40 in contact with the first outer sleeve 35 is pulled by the engagement roller 45 and the idler rollers 46 and 47 on the proximal side. A distal portion of the endless track device 40 in contact with the first outer sleeve 35 is shifted by the engagement roller 45 and the idler rollers 46 and 47 on the distal side. The endless track device 40 is developed widely on the distal side, and turned over after turn around, so that the outer wall 42 of the endless track device 40 in FIG. 2 contacts a wall of the body cavity.

Furthermore, the proximal portion of the endless track device 40 is pulled to turn a proximal portion of the outer wall 42 on the outer side in contact with the wall of the body cavity by 180 degrees, and bent internally in the endless track device 40. Thus, the lumen wall 41 of the endless track device 40 is moved in the distal direction axially. The outer wall 42 of the endless track device 40 is moved in the proximal direction axially, so as to propel the guide assembly 20. Note that the guide assembly 20 can be returned in the proximal direction by turning around the endless track device 40 in the direction reverse to the above direction.

The operation of the guide assembly 20 is described now. When the endoscopic imaging is terminated, the endoscope 10 is removed from the body cavity, for example large intestine. Then the guide assembly 20 is removed from the endoscope 10. The propulsion unit 50 is disengaged from the transmission device 30 before the transmission device 30 is cleaned up by a specialized washing apparatus together with the endoscope 10. After the cleanup, a new propulsion unit 50 is mounted on the transmission device 30 for next examination of imaging.

In FIGS. 7, 8 and 9, the second outer sleeve 52 after removal of the guide assembly 20 is in the set position. The engagement roller 45 and the idler rollers 46 and 47 are set to squeeze the endless track device 40. The transmission device 30 cannot be readily pulled away from the propulsion unit 50. In view of this, the second outer sleeve 52 is rotated in a rotational direction 73 of FIG. 9 on the outside of the endless track device 40 to move the second outer sleeve 52 to the release position of FIG. 10. The idler rollers 46 and 47 are moved by the release spring 55 in an outward direction 74 (direction 71). Thus, the engagement roller 45 is retracted from tight engagement with the idler rollers 46 and 47 by their retraction.

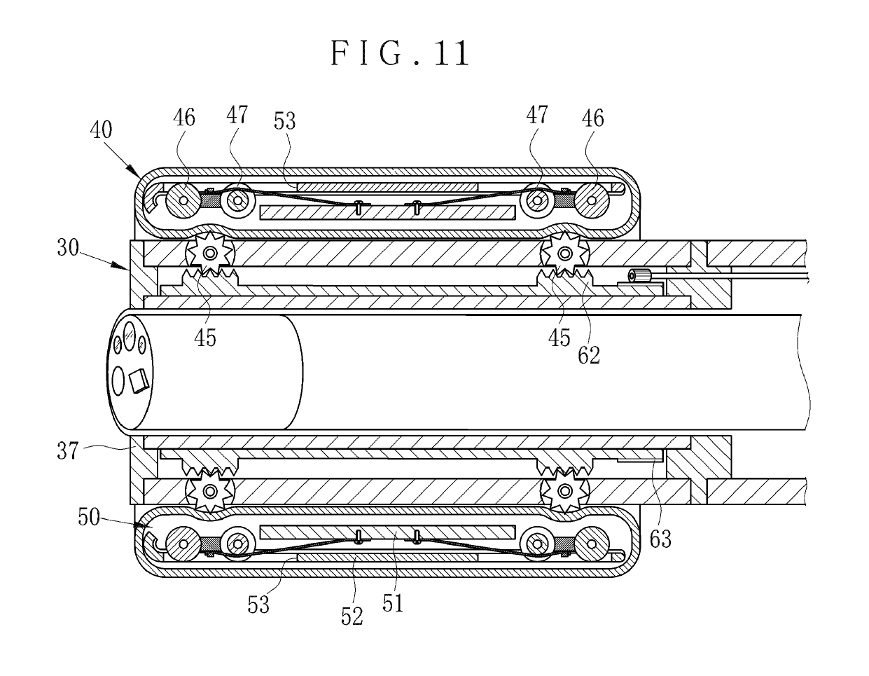

It is possible in FIG. 11 to pull away the propulsion unit 50 easily for removal from the transmission device 30 even with the endless track device 40, because the idler rollers 46 and 47 are retracted from the engagement roller 45 in the propulsion unit 50.

Note that, specifically, the endless track device 40 is prepared in the following manner. At first, a plastic tube having two open ends with flexibility and elasticity is initially formed from a sheet or film of the above-described suitable material. The plastic tube is halfway inserted in a sleeve lumen of the propulsion unit 50 including the second inner sleeve 51 and the second outer sleeve 52. Then a portion of the plastic tube outside the sleeve lumen is bent back externally and extended to cover the periphery of the second outer sleeve 52 of the propulsion unit 50. A first side line of the inserted half of the plastic tube is opposed to a second side line of the bent half to the plastic tube, so that the halves are attached together along the first and second side lines by adhesion, welding or other suitable method. Finally, the toroidal shape of the endless track device 40 is obtained.

Although the present invention has been fully described by way of the preferred embodiments thereof with reference to the accompanying drawings, various changes and modifications will be apparent to those having skill in this field. Therefore, unless otherwise these changes and modifications depart from the scope of the present invention, they should be construed as included therein.

What is claimed is:

1. A guide assembly for an endoscope having a section of an elongated tube for entry in a body cavity, comprising:
a first inner sleeve, having a lumen, for mounting on said elongated tube by receiving entry in said lumen;
a first outer sleeve secured around said first inner sleeve;
an endless track device, having an annular surface, supported around said first outer sleeve, for endlessly moving in an axial direction of said elongated tube in contact with a wall of said body cavity, for propulsion of said elongated tube;
an engagement roller, secured to said first outer sleeve in a rotatable manner, for driving said endless track device endlessly to move;
a second inner sleeve, disposed in an inner space within said endless track device;
an idler roller, secured to said second inner sleeve, and caused to rotate by endless movement of said endless track device;
a setting device, shiftable between a set position and a release position, for pressing said idler roller on said engagement roller when in said set position, to nip said endless track device therewith, and for retracting said idler roller from said engagement roller in an outward direction when in said release position, to enable removal of said endless track device from said first outer sleeve.

2. A guide assembly as defined in claim 1, wherein said setting device includes a second outer sleeve disposed in said inner space within said endless track device and around said second inner sleeve, for rotating relative to said second inner sleeve between said set position and said release position.

3. A guide assembly as defined in claim 2, wherein said first inner sleeve and said first outer sleeve constitute a unit for transmission, and said second inner sleeve, said endless track device and said setting device constitute a toroidal propulsion unit.

4. A guide assembly as defined in claim 2, wherein said second outer sleeve includes:
a pressure portion for pressing said idler roller on said engagement roller when in said set position;
a release portion for enabling said idler roller to move away from said engagement roller when in said release position.

5. A guide assembly as defined in claim 4, wherein said pressure portion is an internal pressure surface of said second outer sleeve, and said release portion is a release opening or recess for receiving said idler roller.

6. A guide assembly as defined in claim 5, wherein said setting device further includes a biasing mechanism for biasing said idler roller away from said engagement roller.

7. A guide assembly as defined in claim 2, wherein a set of said engagement roller and said idler roller is constituted by plural sets arranged equidistantly in a circumferential direction of said first outer sleeve and said second inner sleeve and by plural sets arranged in said axial direction.

8. A guide assembly as defined in claim 7, wherein said idler roller is constituted by two idler rollers disposed on distal and proximal sides relative to said engagement roller in said axial direction.

9. A guide assembly as defined in claim 6, wherein said biasing mechanism is a spring plate retained on said second inner sleeve.

10. A guide assembly as defined in claim 2, further comprising:
a front end ring fitted in a space between distal ends of said first inner sleeve and said first outer sleeve;
a rear end ring fitted in a space between proximal ends of said first inner sleeve and said first outer sleeve.

11. A guide assembly as defined in claim 10, wherein said front and rear end rings are formed together with said first outer sleeve.

12. A guide assembly as defined in claim 2, further comprising:
an input gear, contained in said first outer sleeve, and caused to rotate by an external drive source;
a gear mechanism, disposed between said first inner sleeve and said first outer sleeve, for rotating said engagement roller upon rotation of said input gear.

13. A guide assembly as defined in claim 12, wherein said gear mechanism includes:
a drive sleeve supported around said first inner sleeve in a rotatable manner;
a spur gear, disposed at an end of said drive sleeve, and meshed with said input gear;
at least one tooth set of worm gear teeth, disposed around said drive sleeve.

14. A guide assembly as defined in claim 12, further comprising a rear end ring, fitted in a space between proximal ends of said first inner sleeve and said first outer sleeve, for supporting said input gear in a rotatable manner.

15. A guide assembly as defined in claim 14, further comprising a torque coil structure, connected with said drive source, for applying rotational force to said input gear.

16. A guide assembly as defined in claim 6, wherein said idler roller is constituted by first and second idler rollers having rotational axes substantially parallel with one another;
further comprising a roller support device for supporting said first and second idler rollers in a rotatable manner.

17. A guide assembly as defined in claim 16, wherein said roller support device includes:
first and second side walls for supporting said first and second idler rollers in a rotatable manner;
an arm, disposed between said first and second idler rollers, for connecting said first side wall with said second side wall and supporting said first and second side walls, said biasing mechanism being retained on said arm.
